(19) United States
(12) Patent Application Publication
(10) Pub. No.: US 2005/0205094 A1
(43) Pub. Date: Sep. 22, 2005

(54) Transport aircraft

(75) Inventor: Jean-Michel Cazenave, Seyssins (FR)

Correspondence Address: Linda K. Russell, Suite 1800, 2700 Post Oak Blvd., Houston, TX, 77056, US

(73) Assignee: L'Air Liquide, Societe Anonyme a Directoire et Conseil de Surveillance pour I'Etude et I'Exploita

(21) Appl. No.: 11/021,692

(22) Filed: Dec. 15, 2004

(30) Foreign Application Priority Data
Dec. 15, 2003 (FR) 03 51052

(51) Int. Cl. A62B 7/04; A61M 16/00

(52) U.S. Cl. 128/204.29

(57) ABSTRACT

An apparatus for an oxygen supply system on a transport aircraft. The aircraft, which is suitable for military or civil use, has a crew compartment equipped with at least one quick disconnect mask. The crew compartment mask is capable of being connected to a pressurized oxygen source. The crew compartment also contains at least one mask meant to be permanently worn. The permanently worn mask is capable of being connected to an onboard oxygen or enriched air generator, via a pressure and flow controller. The aircraft also has a transport compartment with at least one mask capable of being connected to either the oxygen source or to the onboard generator.

The present invention relates to transport aircraft, of the type comprising a transport compartment and a crew compartment, and an oxygen supply system comprising an onboard oxygen generator and at least one source of oxygen gas.

The present invention relates more particularly to a transport aircraft architecture designed for military transport, offering great versatility of application and accordingly permitting combined military and civil certification.

For this purpose, according to one aspect of the invention, the crew compartment comprises at least one quick disconnect mask connectable to the oxygen source and at least one permanently worn mask connectable to the oxygen generator.

According to other more particular features of the invention:
the transport compartment comprises at least one mask connectable to the oxygen source and/or at least one mask connectable to the onboard oxygen generator,
the permanently worn mask of the crew compartment is connectable to the onboard oxygen generator via a pressure and flow controller,
the onboard oxygen generator is of the PSA (pressure swing adsorption) type, typically containing a zeolite adsorbent,
the aircraft is designed as a military transport aircraft.

Other features and advantages of the invention will appear from the following description of embodiments, given by way of illustration but not limiting, with reference to the drawings appended hereto, in which:

The single FIGURE schematically shows an oxygen supply installation in a transport aircraft according to the invention.

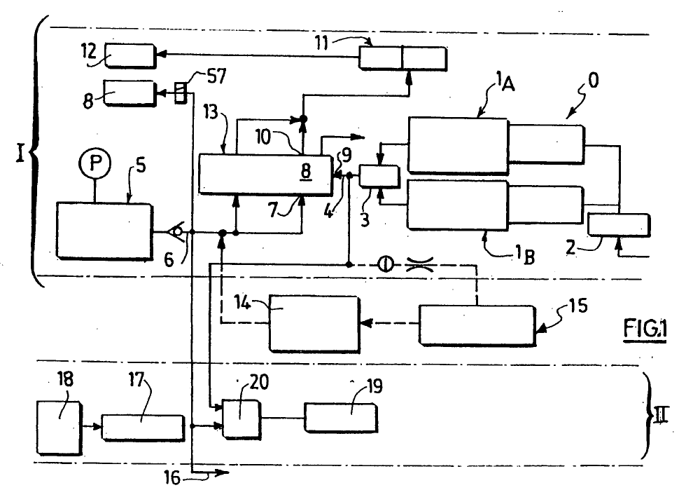

In FIG. 1, an organization of a transport aircraft is schematically shown, comprising in particular a crew compartment (I) and a transport compartment (II) for the transport of equipment and/or persons.

The aircraft is equipped with an oxygen generator, generally denoted by the reference O, of the PSA (pressure swing adsorption) type, (generally denoted by the acronym OBOGS) comprising at least two adsorbers 1A,1B, zeolite adsorbent containing, particularly LiX, advantageously of the type described in patent application FR 03/02708 in the name of the applicant, receiving, via a filter 2, pressurized air from the aircraft engines and operating alternately to supply, via an outlet filter 3, an oxygen-enriched gas mixture in an outlet line 4.

The aircraft further comprises, in the crew compartment I, at least one pressurized oxygen source 5, generally consisting of a plurality of gas cylinders under a pressure above 100 bar connected to an outlet line 6.

According to one aspect of the invention, the outlet line 6 is connectable directly, possibly via an automatic trip device 57, to a set 8 of quick disconnect masks (generally denoted by the acronym QDM) and to an inlet 7 of a distributor/regulator selector 8 having a second inlet 9 connected to the outlet 4 of the oxygen generator O.

According to one aspect of the invention, the regulator/distributor 8, advantageously of the motorized type, comprises an outlet 10 connectable, via a pressure and flow controller 11, to a set 12 of permanently worn military masks possibly combined with a biological/chemical protection unit. The regulator-distributor 8 can be selectively bypassed by a bypass device 13 allowing direct connection of the outlet 6 from the source 5 to the regulator 11.

It may be understood from the above that the crew in the compartment I has at its disposal quick disconnect masks 8 directly connectable to the source 5 as in all civil aircraft crew systems currently in service, thereby permitting certification of the aircraft according to the civil regulations (FAR 25 type) in order to provide logistic transport use comparable to that of a civil transport aircraft, permitting the crew to fly without individual permanent protection units, these units only being employed in case of sudden cabin depressurization or smoke.

According to one aspect of the invention, the crew also has at its disposal, besides the high-pressure oxygen gas storage unit 5, the onboard oxygen generator O which, via the regulator 11 and the adapted masks 12, permits the crew to perform tactical missions while being continuously supplied with oxygen, thereby meeting the various military standards in force (STANAG, MIL-SPEC, etc.).

The oxygen source 5 can be supplemented with portable pressurized oxygen cylinders 14, supplied, from the medium-pressure oxygen outlet 4 of the generator O, via a pressure booster 15 to supplement the source 5, or for individual use, to a medical outlet 16.

For its part, the passenger transport compartment II comprises a set 17 of temporarily worn masks connectable to chemical oxygen generators 18 and/or temporarily worn masks 19 connectable, via a dual inlet control valve 20, to the outlet of the oxygen source 5 or to the outlet 4 of the oxygen generator O, in an arrangement like the one described in document PCT/FR03/50066 in the name of the applicant.

With one or the other of these types of oxygen supply, the transport compartment can be certified de facto in conformity with the above civil aviation standards.

Although the invention has been described in relation to particular embodiments, it is not thereby limited but is suitable for modifications and variants which will appear to a person skilled in the art within the framework of the claims below.

1-7. (canceled)

8. An apparatus which may be used as a transport aircraft, said apparatus comprising:
a) a transport compartment;
b) an oxygen supply system, wherein said oxygen supply system comprises:
1) an onboard oxygen generator; and
2) at least one onboard oxygen source; and
c) a crew compartment, wherein said crew compartment comprises:
1) at least one quick disconnect mask connectable to said oxygen source; and
2) at least one permanently attached mask connected to said oxygen generator.

9. The apparatus of claim 8, further comprising a pressure and flow regulating means, wherein said pressure and flow regulating means is substantially connected to both said oxygen generator and said permanently attached mask.

10. The apparatus of claim 8, wherein said transport compartment comprises at least one mask which is capable of connection to said oxygen source.

11. The apparatus of claim 8, wherein said transport compartment comprises at least one mask which is capable of connection to said oxygen generator.

12. The apparatus of claim 11, further comprising a dual inlet control valve, wherein:
a) one said inlet of said control valve is connected to said oxygen generator;
b) said inlet of said control valve not connected to said oxygen generator is connected to said oxygen source; and
c) the outlet of said control valve is connected to said transport compartment mask.

13. The apparatus of claim 10, further comprising a dual inlet control valve, wherein:
a) one said inlet of said control valve is connected to said oxygen generator;
b) said inlet of said control valve not connected to said oxygen generator is connected to said oxygen source; and
c) the outlet of said control valve is connected to said transport compartment mask.

14. The apparatus of claim 8, wherein said oxygen generator comprises a pressure swing adsorption oxygen generator.

15. The apparatus of claim 8, wherein said transport aircraft comprises a military transport aircraft.
